(19) United States
(12) Patent Application Publication
(10) Pub. No.: US 2010/0240579 A1
(43) Pub. Date: Sep. 23, 2010

(54) DETECTION OF CELL SURFACE BINDING MOLECULES USING A PHAGE DISPLAY BLOCKING ASSAY

(75) Inventor: Sara Louise Cosby, Belfast (GB)

Correspondence Address: JENKINS, WILSON, TAYLOR & HUNT, P. A., Suite 1200 UNIVERSITY TOWER, 3100 TOWER BLVD.,, DURHAM, NC, 27707, US

(73) Assignee: THE QUEEN'S UNIVERSITY OF BELFAST, Belfast (GB)

(21) Appl. No.: 12/666,412

(22) PCT Filed: Jun. 26, 2008
(86) PCT No.: PCT/GB2008/050503

§ 371 Date: May 24, 2010

(30) Foreign Application Priority Data
Jun. 26, 2007 (GB) 0712379.7

(51) Int. Cl. A61K 38/02; C12Q 1/70; G01N 33/53; A61P 31/12

(52) U.S. Cl. 514/3.7; 435/5; 435/7.1

(57) ABSTRACT

The present invention relates to the use of a phage blocking assay to determine unknown binding molecule present on or in the surface of a cell, a non-infectious moiety, a bacteria, a virus, or another pathogen. In particular embodiments, the invention relates to the identification of unknown receptors on a cell or virus involved in infection. Further, it relates to the use of said binding molecules, for example virus binding molecules or cellular receptors and binding members with binding specificity to said binding molecules, for example antibodies, in methods of therapy.

### FIELD OF INVENTION

The present invention relates to the use of phage to determine unknown binding molecules present on or in the surface of a cell, a non-infectious moiety, a microarray, a bacteria, a virus, or another pathogen. In particular embodiments, the invention relates to the identification of unknown receptors on a cell or on a virus which are involved in infection. Further, it relates to the use of said binding molecules, for example on a virus or cellular receptors and binding members with binding specificity to said binding molecules, for example antibodies thereto, in methods of therapy.

### BACKGROUND

Knowledge of binding molecules present on the surface of cells or pathogens, which allow interaction between cells in an organism or, between pathogens and cells, for example receptors which mediate infection of a cell by a virus, is important to allow an understanding of processes within organisms. In particular, knowledge of interactions between host cells of organisms and invading pathogens is important to allow elucidation of new drug targets.

For known binding molecules associated with a biological function, such as known receptors involved in infection, using the known receptor, immune responses can be provoked in animals to provide antibodies specific to the receptor. In addition, other assays utilising the known receptor can be performed. As an alternative to animal methods, methods such as phage display can be utilised to generate antibodies to the known receptor. Expression of functional antibodies (Abs) or antibody fragments on the surface of filamentous phage provides a method to select antibodies from libraries on the basis of the antigen-binding behaviour of individual clones (McCafferty et al., 1990). Antibody fragments expressed on the surface of a bacteriophage particle such as, for example, fd or M13 and its encoding genes are fused in frame to one of the phage coat proteins, cloned into a vector and packed as a phage particle (reviewed in Hoogenboom 2005). Monovalent or multivalent display of an antibody depends on the type of anchor protein and display vector used. The monovalent display is convenient for selecting antibodies of higher affinity while multivalent display is used for selecting antibodies that mediate receptor-mediated endocytosis (Poul and Marks, 1999), panels of antibodies for target discovery (O'Connell et al., 2002) and rapid selection of antibody-antigen pairs on the tip of the phage (de Wildt et al., 2002). Phage display is simple to use and highly versatile as the selection process can be adapted to many specific conditions such as selection on whole cells and tissues of humans or animals.

Whilst existing techniques can be utilised to generate antibodies to known receptors, there remains a need to be able to identify receptors which, for example, mediate invasion of cells by pathogens and to identify and characterise unknown binding members present on the surfaces of cells and/or pathogens which mediate biological events following interaction via the binding molecule.

The use of mAb libraries, raised against specific cell types, to successfully identify a receptor for Measles Virus (MV) has been reported (Naniche et al., 1992). However, this method is time consuming, expensive and the mAbs generated may not be fully representative.

### SUMMARY OF INVENTION

The inventor has determined a novel method to identify unknown binding molecules, in particular binding molecules on the surface of cells and viruses.

In particular, the present invention relates to a new technique using a recombinant antibody library to provide a novel route to identify unknown binding molecules/ligands on the surface of a cell, non-infectious particle, or pathogen, particularly binding molecules/ligands which mediate infection, and more particularly which mediate infection of cells by a virus, such as, for example, measles.

According to a first aspect of the present invention there is provided a method for identifying an unknown binding molecule present on an infective agent or a cell capable of being infected, the method comprising the steps of:
providing a test surface including an unknown binding molecule which mediates infection,
exposing the test surface, for example a cell membrane or a virus coat, to at least one phage wherein the phage has at least one displayed peptide on the phage surface, preferably wherein said displayed peptide is an antibody or a fragment thereof,
providing conditions which permit binding of a displayed peptide to a binding molecule of the test surface when the displayed peptide has binding specificity to a binding molecule of the test surface;
providing conditions which allow infection,
determining the level of infection,
   wherein, when the amount of infection in the presence of the phage is decreased relative to a control, it is indicative that the phage is bound to a binding molecule which mediates infection,
using bound phage to select the binding molecule in or on the test surface to which the phage binds and
identifying said binding molecule.

The use of a phage to block binding to a test surface allows determination of “blocking” by the phage and indicates the phage is binding to a receptor necessary for infection.

Suitably, in embodiments of the invention, the test surface can be a surface of a cell which is capable of being infected or the surface of an infective agent capable of infecting the cell.

Suitable conditions under which a phage displaying a peptide with binding specificity to a binding molecule of a test surface could bind to such a binding molecule would be known to those of skill in the art. These are likely to be similar conditions as presently used in relation to panning protocols for phage display. Suitably, after such conditions have been provided the test surface may be washed with a detergent solution, for example Triton X, to remove non-specific binding. If the phage clone expresses a displayed peptide that can interact with a binding molecule, as the binding molecule is immobilized on the test surface, the displayed peptide of the phage will form a stable complex with the binding molecule and be retained.

Suitably, when the test surface, including an unknown binding member, is a cell membrane of a cell which can be infected, in the step of providing conditions which allow infection, the test surface, can be contacted with an infective agent. In embodiments of the method when the test surface which includes an unknown binding member is an infective agent, the test surface can be brought into contact with a cell which may be infected.

Suitably a binding molecule of a test surface may be provided in the surface, on the surface or attached to the surface by, for example, a linker.

Suitably the control is the assay when the test surface is exposed to no phage or to a phage which expresses a peptide known not to bind to a binding molecule on the test surface. In particular embodiments a peptide displayed on the phage being used as a control can have binding specificity to ubiquitin. As a further control, a binding member which binds to the test surface, but which does not block the interaction being considered can be provided for comparison.

In a preferred embodiment, the test surface is the surface of a cell, for example a cell membrane, or the surface of a infective agent or pathogen, for example a virus coat. The methods of the present invention may be applied to a cell membrane preparation as a test surface and are not limited to the use of intact cells.

Where the unknown binding molecule source is a whole cell line or a tissue homogenate, sub libraries of phage displayed peptides, for example antibody, may be provided which should recognise a high percentage of binding molecules in that preparation.

In particular embodiments the step of identifying includes characterisation of at least one of the binding molecule that mediates infection of a cell.

Suitably, identification/characterisation of the binding molecule may be performed by any one of the methods comprising; immunoprecipitation, Sodium Dodecyl Sulfate Polyacrylamide gel electrophoresis (SDS-PAGE), 2D gel electrophoresis, mass spectrometry e.g. Matrix Assisted Laser Desorption Ionisation-Time of Flight (MALDI-TOF) or Western blot. The skilled person would understand the method of characterisation which should be employed to test for and determine characteristics of a binding molecule, for example to determine whether the binding molecule comprises amino acids, lipids or sugars, the weight of the binding molecule, the charge of the binding molecule and the like. In specific embodiments of the methods of the present invention, the step of characterising at least one of the binding members or a peptide with binding specificity to the binding members may also include determining the nucleic acid sequence encoding said binding member or the amino acid sequence of the binding member when the binding member is proteinaceous or a peptide with binding specificity to said binding member.

In particular embodiments, the infective agent can be a virus. In such embodiments the method can comprise the steps:
providing a surface of a cell, capable of being infected by a virus of interest, as a test surface,
exposing said cell test surface to at least one phage wherein the phage displays at least one displayed peptide, preferably wherein said displayed peptide is an antibody or a fragment thereof, under conditions which permit binding of the displayed peptide to a binding molecule on the test surface when the displayed peptide has binding specificity to the binding molecule on the test surface,
contacting the cell which may be capable of being infected, which has been exposed to the at least one phage, to the virus of interest, under conditions suitable to allow infection of the cell,
determining the level of infection,
   wherein, when the amount of infection in the presence of the phage is decreased over a control it is indicative that the phage is bound to a binding molecule which mediates infection of a cell,
using the peptide displayed on the phage to select the binding molecule in or on the cell surface to which the phage binds and
identifying said binding molecule on the cell surface.

As will be appreciated, in an alternative embodiment to determine a binding molecule which mediates infection or attachment of, for example, a cell to a virus, wherein the binding molecule is present on a virus of interest, the test surface (virus) can be
exposed to at least one phage wherein the phage displays at least one peptide,
conditions which permit binding of a displayed peptide with binding specificity to a binding molecule on the test surface can be provided,
the virus and the cell capable of being infected can be contacted to allow for infection,
then infection of a cell by the virus can be determined.

Should the virus not be able to infect the cell or show decreased infection, the phage can be used to identify the binding molecule of interest on the virus which has been blocked.

Suitably, in particular embodiments of the method to determine a binding molecule which mediates infection of a cell, the step of determining the level of infection can be determined by measuring infection of a cell with the infective agent. In such embodiments infectivity titrations or immunofluorescence can be used to determine infection.

Suitably, in particular embodiments of the invention, the at least one phage can be provided in a pool of phage where each or groups of phage in a respective pool display different peptides. In such embodiments, when a pool of phage leads to a decrease in the level of infection or an event, the pool can be subdivided and the method repeated such that a smaller pool of given phage can be tested. Suitably, a selection and/or an amplification step may also be utilised. Subdivision of pools of phage can be repeated until the identification of a phage expressing a peptide capable of inhibiting infection or another biological event is provided.

In embodiments of the methods of the invention, following the step of exposing the test surface to phage displaying peptides, under conditions which allow the binding of peptides displayed by the phage to the binding molecule, non-bound phage may be removed. In such an embodiment, phage may display antibodies or fragments thereof and be exposed to a test surface under conditions which allow those antibodies with binding specificity to binding molecules on the test surface to bind the binding molecules. Non-bound phage may then be removed by, for example, washing of the test surface. Where the binding molecule of the test surface is bound by a peptide displayed by a phage, infection is blocked during the step of contacting the test surface with an infective agent. In embodiments the peptide of the phage inhibiting infection can then be identified and used to isolate the unknown receptor. For example, the peptide displayed by the “blocking phage” may be used as a selective ligand in an affinity purification strategy for the unknown receptor. Alternatively, the phage may be selected using antibodies with specificity to the phage or reporters of the phage, for example fluorescent molecules bound to the phage. The unknown receptor may then be identified using, for example mass spectroscopy.

Alternatively, in embodiments to determine an unknown binding molecule on the surface of a pathogen, for example a protein of a virus coat, a phage displaying peptides can be brought into contact with the surface of the pathogen under conditions which would allow binding of a phage displaying a peptide with suitable binding specificity to the binding molecule of the pathogen. Suitably, after binding of such phage with binding specificity, the unbound phage may be removed, and the pathogen brought into contact with a cell to determine if infection can occur. In cases where infection of the cell is blocked, it is indicative that the peptide displayed by the phage is bound to a binding molecule on the pathogen which mediates infection. As above, the peptide of the phage or the phage inhibiting infection may then be used to isolate the unknown binding molecule which mediates infection and/or attachment, for example using the peptide as a selective ligand in a purification strategy for the unknown receptor or utilising antibodies to the bound phage. The unknown receptor may then be identified using, for example mass spectroscopy.

In particular embodiments the virus can be a portion of a virus, for example virus coat component.

A virus used in the methods of the invention, either as the test surface or the infectious agent may be any virus of interest, for example a virus which binds to and/or infects a cell being tested and which may cause a disease state to develop. In preferred embodiments, the virus of interest can be any one of the group comprising the following; Measles Virus (MV), phocine distemper virus (PDV), dolphin morbillivirus (DMV) and canine distemper virus (CDV).

The inventor has further realised the potential of the above technique, wherein the interaction blocked by the peptide expressed by the phage does not lead to infection of a cell, but to another detectable/measurable event, in particular a detectable/measurable biological event for example cell migration, differentiation, apoptosis or the like. In view of this, the method can be utilised to determine binding molecules involved in binding of non-infectious particle/molecules to a test surface.

Accordingly, a second aspect of the present invention provides a method for identifying a binding molecule on a test surface, the method comprising the steps of:
providing a test surface;
providing at least one phage expressing at least one displayed peptide, on the phage surface;
exposing the test surface to the at least one phage under conditions which permit binding of a displayed peptide to a binding molecule on the test surface when the displayed peptide has binding specificity to a binding molecule on the test surface;
contacting the test surface with an agent which interacts with the test surface via an unknown binding molecule wherein said agent can be detected by reporter means;
determining the level of the agent bound to the test surface using the reporter means;
   wherein, when the level of the agent bound to the test surface in the presence of a phage is decreased relative to a control, it is indicative that the phage is bound to a binding molecule which mediates binding to the agent;
   using the bound phage to select the binding molecule in or on the test surface to which the phage binds and
identifying said binding molecule.

Suitably said reporter means may include, for example, a fluorescent reporter molecule or a radioactive label provided on the agent or the reporter means can include an antibody capable of binding to the agent.

Suitably in embodiments, the reporter means which allows detection of bound agent can be a biological event, for example cell migration, differentiation, virus replication or the like.

In embodiments of the method for identifying a binding molecule on a surface of a cell or pathogen wherein the reporter means is a detectable/measurable biological event, the method comprises the steps of:
providing a test surface;
providing at least one phage expressing at least one displayed peptide, wherein the displayed peptide is preferably an antibody or fragment thereof, on the phage surface;
exposing the test surface to the at least one phage under conditions which permit binding of a displayed peptide to a binding molecule on the test surface when the displayed peptide has binding specificity to a binding molecule on the test surface;
contacting the test surface with an agent which interacts with the test surface via an unknown binding molecule and causes a biological event,
determining the level of the biological event;
   wherein, when the level of the biological event in the presence of a phage is decreased relative to a control, it is indicative that the phage is bound to the binding molecule which mediates the biological event;
   using the peptide provided on the phage to select the binding molecule in or on the test surface to which the phage binds and
identifying said binding molecule.

In particular embodiments the agent can be selected from a protein, a proteinaceous moiety, a lipid, a carbohydrate, a virus or portion thereof, a bacterium or portion thereof, or fungi or a portion thereof.

Whilst previous use of phage display has allowed antibody production against known antigens, the present methodology provides for the identification of previously unknown antigens.

The novel use of the phage libraries in “blocking assays” as described by the first and second methods of the invention provides a convenient way to identify unknown antigens, in particular receptors/binding molecules present in, on or attached to a surface, for example, but not limited to the surface of a cell, a pathogen or non-infectious moiety. In particular embodiments, the method could be used to identify a protein on a microarray.

In particular embodiments, the method further includes the step of characterising the binding molecule identified in or on the test surface.

Suitably, in particular embodiments, the at least one peptide on a phage surface expressed by a phage can be encoded by a nucleic acid sequence from an antibody fragment library. In certain embodiments, the antibody fragment library can be constructed from at least one of:
(i) Ig variable region genes derived from the B cells of an immunized donor;
(ii) B cells of a non-immunized donor;
(iii) a combination of germline V genes and synthetic oligonucleotide sequences encoding random CDRs;
(iv) a fully synthetic library; and
(v) the Tomlinson I and J phage display libraries.

The Tomlinson I and J phage display libraries are so called “naïve” or “single pot” phage-antibody library in phagemid/scFv format. Both I and J libraries differ from antibody libraries derived from immunized animals or immune donors and antibody libraries from non-immunized donors. In these types of library, the antibody can only be obtained against the set of antigens to which an immune response was induced (Hoogenboom et al., 1998). The Tomlinson I+J libraries are based on a single human framework V_{H} and V_{K} with side chain diversity incorporated at the positions in the antigen binding site that makes contacts with antigen in known structures.

Suitably the binding molecule may be a binding molecule which mediates at least one of virus attachment to a cell, virus entry into a cell, bacterial attachment to a cell, bacterial entry into a cell, bacterial like agents attachment to a cell, fungal attachment to a cell, cell differentiation, cell migration, cytokine release, cell apoptosis, or a change in cellular morphology.

In particular embodiments the binding molecule can be attached to the cell membrane in any suitable form, for example, via a tether, by including a membrane anchoring portion, by including a transmembrane portion or by any other suitable means.

In preferred embodiments, a test surface may be a membrane of a mammalian cell. In further specific embodiments, a test surface can be any one of the group comprising the following; a neuroblastoma cell, for example, but not limited to, SHSY5Y, a Vero cell, a neuronal cell for example, but not limited to NT2. In particular embodiments, a test surface can be provided in vitro, for example a microarray. The test surface need not be a whole cell.

As may be appreciated, in addition to identifying a binding molecule present on a cell membrane or another agent capable of interacting with a cell, the peptide expressed by the phage, preferably an antibody fragment such as, but not limited to a scFv, which binds to the binding member of interest can be identified and optionally characterised.

The use of phage display technology is advantageous as it provides a rapid, adaptable way, without the use of animals, to determine peptides, for example antibodies or fragments thereof, which are capable of binding to binding molecules and using the methods of the present invention, the corresponding binding molecules themselves in, on or attached to a test surface.

Given the teaching provided herein, the present methods may be utilised to determine binding molecules involved in infection and also for binding molecules involved in non-infectious diseases, for example cancer, allergic, genetic and neurogenerative disorders. These newly identified receptors/binding molecules may be useful as drug targets or for use in medicaments or diagnostics. The phage or peptides displayed on the phage which bind to the receptor/binding molecule may also be used in medicaments or diagnostics.

The present methods are advantageous over animal methodologies in that DNA sequences coding for a selected peptide, displayed on a phage, for example an antibody displayed on a phage, can be easily mutated to produce peptides, for example antibodies with higher affinity (stronger binding) to the target (unknown receptor/binding molecule) than the originally selected peptide/antibody displayed on the phage. This may be useful to allow the unknown binding molecule on the surface of the cell or pathogen to be identified and characterised or it may be useful, for example if the peptide is to be utilised for therapeutic purposes, i.e. to block infection or binding interaction.

In addition, to determining antigens, for example, unknown binding molecules on a test surface, the present invention may be used to identify the unknown peptide attached to the phage capable of binding to and/or blocking the binding to the unknown antigen. Thus, the present invention provides for the determination of two unknowns i) an unknown target binding molecule (receptor) and ii) an unknown peptide capable of binding to, i.e. with binding specificity to, the target binding molecule.

In particular embodiments, the method of the invention may comprise the further step of using the binding molecule identified on the test surface to identify a peptide with binding specificity to the binding molecule. Optionally, the identified binding molecule may be used to increase the binding specificity of a peptide with binding specificity to the binding molecule. As will be appreciated, having identified the unknown binding molecule on the test surface, conventional methods to generate antibodies or aptamers to said binding molecule may be utilised.

Thus, according to a third aspect of the invention, there is provided a method for identifying a peptide with binding specificity to an unknown binding molecule present on a test surface capable of mediating infection or another event present on a test surface, wherein said method comprises the steps of:
providing a test surface,
providing at least one phage expressing at least one displayed peptide, preferably an antibody or a fragment thereof, on the phage surface;
exposing the test surface, for example a cell membrane or a virus coat, to the at least one phage under conditions which permit binding of a displayed peptide to a binding molecule on the test surface when the displayed peptide has binding specificity to a binding molecule on the test surface;
contacting the test surface with an agent which interacts with the test surface via an unknown binding molecule wherein said agent can be detected by reporter means,
determining the level of the agent bound using the reporter means;
   wherein, when the level of the reporter means in the presence of a phage is decreased relative to a control test surface, it is indicative that the phage is bound to the unknown binding molecule which mediates the event, and identifying the peptide displayed by the given phage with binding specificity to the binding molecule of the test surface.

In embodiments, the test surface can be selected from the surface of a cell, a pathogen or a non-infective moiety, for example a microarray.

Suitably, the binding molecule can mediate infection of a cell by a bacterium, virus, fungi or other agent. In such embodiments, the method can include the steps of:
providing the surface of a cell which may be infected or the surface of an infective agent capable of infecting the cell as a test surface,
exposing the test surface, for example a cell membrane or a virus coat, to at least one phage wherein the phage expresses at least one displayed peptide, preferably wherein said displayed peptide is an antibody or a fragment thereof, on the phage surface under conditions which permit binding of a displayed peptide to a binding molecule on the test surface when the displayed peptide has binding specificity to a binding molecule on the test surface;
contacting the cell which may be infected and infective agent capable of infecting the cell under conditions suitable to allow infection of the cell,
determining the level of infection,
   wherein, when the amount of infection in the presence of the phage is decreased relative to a control test surface, it is indicative that the phage is bound to a binding molecule which mediates infection of a cell,
identifying the peptide provided on the phage with binding specificity to the binding molecule in or on the test surface to which the phage binds.

In embodiments of the invention, a peptide identified as binding to an unknown binding molecule provided by a test surface can be used in assays to identify modulating agents which specifically bind to the unknown binding molecule. Such assays comprise the steps: contacting the identified peptide or the phage displaying the peptide, as identified using the methods of the invention, with binding specificity to the unknown binding molecule, (i.e. capable of “blocking” binding to the unknown binding molecules) in the presence and absence of a putative modulating agent under conditions which would allow binding between the peptide with binding specificity to the unknown binding molecule and the unknown binding molecule in the absence of the putative modulating agent and determining the level of binding of the peptide to the binding molecule in the presence and absence of the putative modulating agent, wherein when the putative modulating agent decreases the level of displayed peptide bound by the binding molecule the putative modulating agent is considered to have binding specificity to the unknown binding molecule. In embodiments, the peptide displayed by the phage may be provided alone, or conjugated to another molecule, for example a tag or marker molecule. Suitably, the phage displaying the peptide with binding specificity to the unknown binding molecule, the peptide alone or the peptide conjugate may be tagged to allow detection of binding to the unknown binding molecule to be measured. The step of determining the level of interaction may be performed by a range of means as would be generally used by a person of skill in the art to determine competitive binding, for example where the peptide is tagged, the amount of tag bound to the test surface can be determined.

Where the putative modulating agent competes for binding of the unknown binding molecule with the peptide, it is indicative that the putative modulating agent has binding specificity to the unknown binding molecule. Such an assay can be useful to screen libraries of antibodies, antibody fragments, small molecule libraries, peptide libraries and the like for agents able to bind the unknown binding molecule and then to determine if such agents can cause a biological event associated with said unknown receptor or inhibit such an event.

In particular embodiments the infective agent can be a virus or at least a portion thereof, for example a protein of the coat of the virus. In particular embodiments an unknown binding molecule can be proteinaceous, lipid, and/or a carbohydrate moiety component of the cell membrane or a portion of an outer surface, of an infective agent, for example a protein of a virus coat.

Suitably, in preferred embodiments of the method, the peptide expressed on the surface of a phagemid can be an antibody fragment. In particular embodiments, an antibody fragment can be at least one of the group comprising the following: Fab, Fab′, F(ab′)₂, scFv, dsFv, Fv, dAb, Fd, and diabodies.

In a specific embodiment the scFv is a monoclonal scFv.

Unless the context demands otherwise, reference to a peptide or peptides of the invention encompasses derivatives and fragments of a peptide(s) identified by the method of the third aspect of the invention.

Fragments and derivatives for use in the invention suitably retain binding activity to the binding molecule attached to the cell membrane, which binding molecule can mediate infection of a cell by a virus.

Suitably for phages identified as having peptides with binding specificity to an unknown binding molecule which mediates a biological event, the nucleic acid encoding the peptide displayed on the phage can be recloned from the phage into an expression vector to provide peptide, for example antibody which can be used for affinity purification of the unknown binding molecule. In particular embodiments, mutations can be introduced.

In embodiments of the methods of the invention, the methods can further comprise the steps of:
bringing a phage into contact with a test surface such that a phage, which expresses a peptide on the phage surface wherein said peptide which has binding specificity for a binding molecule of the test surface, binds to the binding molecule;
separating phage which bind to the test surface from phage not bound to the test surface, and
optionally, repeating these steps.

In particular embodiments, the method of separating phage which bind to a test surface from phage not bound to a test surface is performed by washing the test surface, for example washing the surface with a buffered detergent solution and/or high salt solutions under suitable conditions. As will be appreciated, suitable conditions will depend on the test surface, for example where the test surface is a cell membrane, the conditions should not cause lysis of the cell. This can be advantageous to improve the selection of phage which are cell membrane/specific. In suitable embodiments the step of separating phage separates the phage on the basis of being able to block a biological event, for example infection of a cell.

In embodiments of the invention, the methods further comprise infecting a plurality of bacterial cells with a selected phage. Preferably the selected phage is the phage identified as displaying a peptide which binds to the unknown binding molecule of the test surface which mediates an event. This can be advantageous to amplify the numbers of a given phage. In preferred embodiments, the bacterial cells are TG1 E. coli cells.

Preferably, amplification and/or selection steps are repeated, at least twice, more preferably at least three times, at least four times, or at least five times. In particular embodiments the selection and/or the amplification steps are repeated at least ten times. Suitably, a helper phage can be utilised, as would be appreciated in the art.

Suitably in the blocking portion of the assay, whether blocking is provided by phage to inhibit infection (first aspect) or provided by the phage to inhibit an event (second aspect) or to compete with the binding of putative modulating agent to the binding molecule, phage used in the method may be provided in a number of pools with phage displaying different peptides being provided in different pools. Those pools of phage which inhibit infection, inhibit an event from occurring or inhibit binding of a putative modulating agent can be progressively subdivided into smaller pools and the methods repeated. As the pools of phage become progressively smaller and the methods repeated, it will be possible to determine a pool containing a single colony of phage which display the same or similar peptide which inhibit infection or an event from occurring or binding of a putative modulating agent to an unknown receptor. This colony can then be used to identify the unknown binding member, for example a receptor.

In specific embodiments, a phage for use in the invention is KM13. Suitably, in particular embodiments the phage further comprises an ampicillin resistant pIT2 vector.

In particular embodiments, a test surface for use in the methods of the invention comprises at least 5%, at least 10% of the material of a whole cell, more suitably at least 20%, at least 30%, at least 40%, at least 50%, at least 60%, at least 70%, at least 80%, at least 90%, at least 95%, at least 99%, at least 100% of the material of a whole cell. Such a preparation allows determination of the presence of proteinaceous, lipid or carbohydrate ligands.

In further embodiments, where, for example, it is known the binding molecule/ligand is proteinaceous, a membrane preparation for use in the methods of the invention comprises at least 10% of the proteinaceous material of a whole cell, more suitably at least 20%, at least 30%, at least 40%, at least 50%, at least 60%, at least 70%, at least 80%, at least 90%, at least 95%, at least 99%, at least 100% of the proteinaceous material of a whole cell.

In specific embodiments, the membrane preparation can be bound to a solid support. In yet another embodiment, the membrane preparation can be a whole test cell bound to a solid support.

Peptides displayed by a phage, for example antibodies or fragments thereof, may additionally possess a binding tag. Usefully, in such embodiments, the binding tag can include a myc tag or a His tag. This provides for simpler purification of a phage and corresponding binding molecule bound to the peptide expressed by the phage. This may be used to detect binding of the phage to a cell and act as reporter means.

Alternatively, in particular embodiments, an antibody specific to a coat protein of the phage can be used to bind phage. For example, in particular embodiments, wherein the phage is M13, an antibody with binding specificity to M13 could be used to select a phage to allow identification of the unknown binding molecule of a test surface, for example a cell membrane or a virus.

Binding molecules, as identified by the methods of the first, second and third aspects of the present invention, can have utility in methods, uses and medicaments. For example, the binding molecules may be used in diagnostic kits and assays. In particular examples the binding molecules may be useful in medicaments, for example in cancer treatment, where a biological event associated with cancer is being detected, for the suppression of infection or other disease states or conditions associated with the agent which binds to the test surface or the event being detected. In particular embodiments, providing said binding molecules/ligands to a body, for example a human body, may bind to and inactivate infectious agents, for example, virus or bacteria within the body.

Peptides, expressed on the surface of phage, as identified by the method of the third aspect of the present invention, can have utility in methods, uses and medicaments. For example, the peptides can be used in diagnostic kits and assays. In particular embodiments the peptides may be used in medicaments for the treatment of particular diseases associated with the agent being applied to the test surface or the event being detected, for example the peptides may be useful for cancer treatment, for the suppression of infection or in relation to other diseases or conditions. Said peptides, for example antibodies, can have utility in the inhibition of viral infections such as measles.

Accordingly, a fourth aspect of the present invention provides a method of inhibiting infection of a cell by an infective agent, for example, but not limited to a virus, wherein the method comprises administering a binding molecule, as identified by the first or second aspects of the present invention, or a peptide, as identified by a the third aspect of the present invention, to a cell.

Accordingly, a fifth aspect of the present invention provides a method of treating a disease in a subject, said method comprising administering a binding molecule, as identified by the methods of the first or second aspects of the present invention, or a peptide, as identified by the third aspect of the invention, to said subject. In certain embodiments the subject can be a mammal, typically a human.

In a sixth aspect of the present invention, there is provided the use of a binding molecule, as identified by the first or second aspects of the present invention, or a peptide, as identified by the third aspect of the invention, in the preparation of a medicament for the treatment of a viral-based disease.

According to a seventh aspect of the present invention, there is provided nucleic acids that encode a binding molecule, as identified by methods of the first and/or second aspects of the invention and/or a peptide as identified by a method of the third aspect of the invention respectively.

According to a further aspect of the present invention, there is provided a kit for use in the methods of the present invention comprising sub-library of phage displaying peptides with binding specificity to the binding molecules provided on a selected cell type or tissue preparation. In embodiments of the invention the selected cell type can include at least one of neuronal cells, NT2 cells, neuroblastoma cells, SHSY5Y cells, Vero cells, epithelial cells, mouse cells, rat cells, human cells. In particular embodiments neuronal cells or epithelial cells of the kit are human.

As will be understood by a person of skill in the art, for convenience and where suitable, a phagemid may be used in instances where reference is made to phage.

Preferred features and embodiments of each aspect of the invention are as for each of the other aspects mutatis mutandis unless context demands otherwise.

An embodiment of the present invention will now be described with reference to the following examples which are provided for the purpose of illustration and are not intended to be construed as being limiting on the present invention wherein:

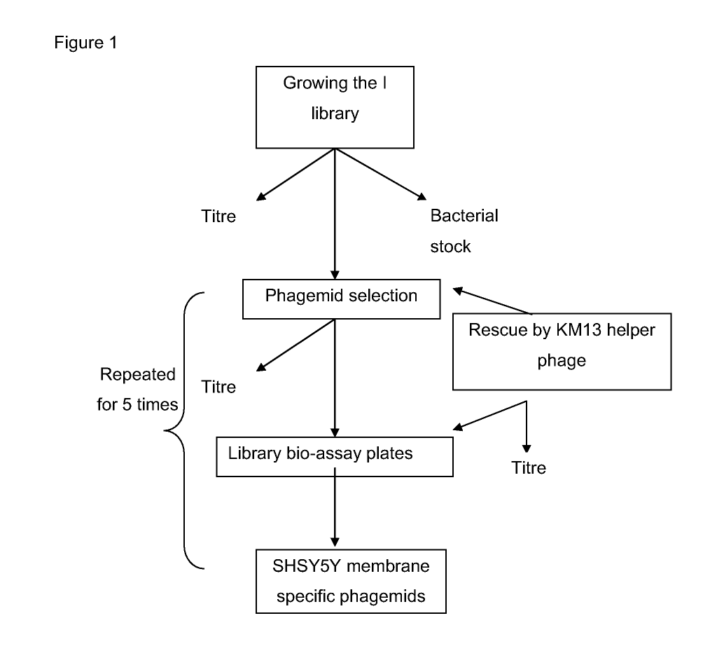

FIG. 1 is a schematic diagram illustrating the key steps involved in production of phagemids from Tomlinson I library as described in the present invention;

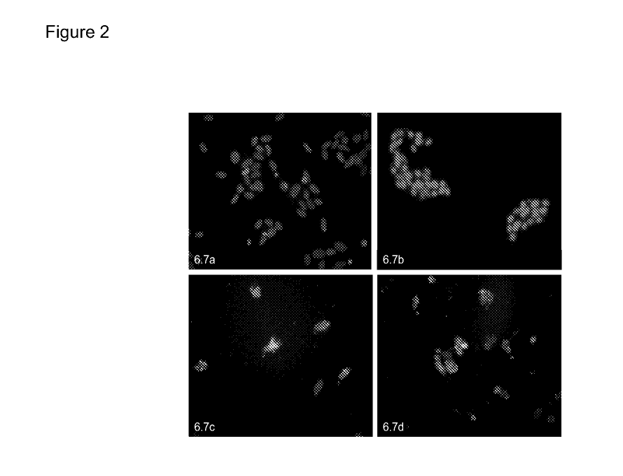

FIG. 2 illustrates a phage blocking assay using immunofluorosecence staining wherein FIG. 2a shows the infection of WT MV (10⁻² dilution) on SHSY5Y cells at 24 hpi after blocking with phage 4.7.3.1; FIG. 2b shows the infection of WT MV (10⁻² dilution) on SHSY5Y cells at 48 hpi after blocking with phage 4.7.3.1; FIG. 2c shows the infection of WT MV (10⁻² dilution) on SHSY5Y cells at 48 hpi in the presence of anti-ubiquitin phagemid control and FIG. 2d shows the infection of WT MV (10⁻² dilution) on SHSY5Y cells at 48 hpi with no phage;

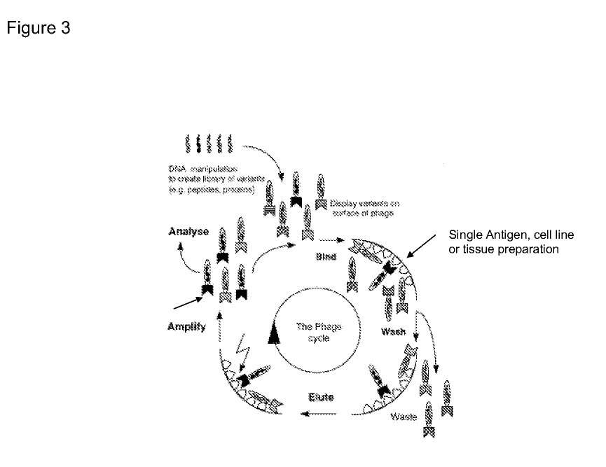

FIG. 3 illustrates panning of phage antibodies to select those binding to a specific antigen, cell line or tissue wherein phage antibodies which after several rounds of panning bind to the preparation are collected and grown up—where used against a single antigen they can be used directly, but if used against a cell line, they comprise a cell panel and can be used in the blocking assay of the methods of the invention to identify unknown molecules on or in the cell; and

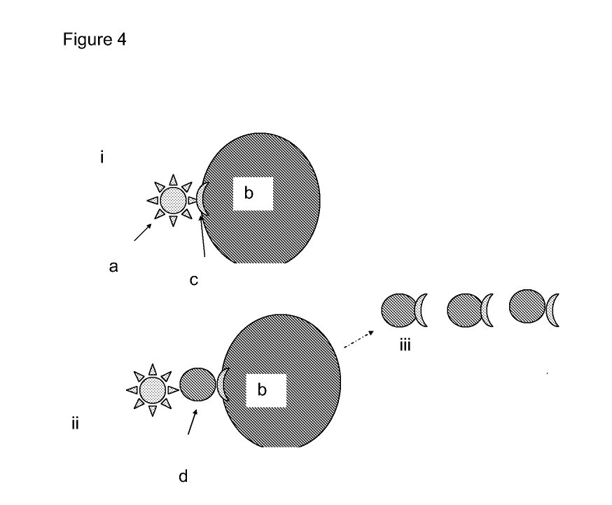

FIG. 4 illustrates screening for blocking antibodies and identification of cell surface binding receptor/molecule (a) is virus, bacteria, a cell or other particle or molecule binding to a test surface (b), (c) is an unidentified cell receptor/binding molecule on the test surface, ii indicates (d) a phage antibody selected from a sub library which blocks attachment of (a), iii shows use of phage antibody (d) to pull out and purify the unknown receptor/binding molecule which can be identified using mass spectroscopy and which may be used as a target from drug therapy and/or may be used a disease biomarker.

### DETAILED DESCRIPTION

By the term “viral disease” is meant those diseases which are caused or induced by pathogenic viruses belonging to either the category of DNA viruses or the category of RNA viruses. Examples of such viruses include negative strand RNA viruses e.g. paramyxovirus (e.g. measles and Sendai), orthomyxovirus (e.g., influenza virus), rhabdovirus (e.g., rabies and vesicular stomatitis virus), positive strand RNA viruses such as cornoaviruses, picornavirus, flaviviruses and alphavirus, single stranded DNA viruses, such as parvoviruses (e.g., adeno-associated viruses) and double stranded DNA viruses including adenovirus, herpesviruses (e.g., Herpes Simplex virus types 1 and 2, Epstein-Barr virus, cytomegalovirus), papovaviruses and poxviruses (e.g., vaccinia, fowlpox and canarypox). Other viruses include, for example, Norwalk viruses, reoviruses, hepadnaviruses, and hepatitis viruses e.g. hepadnaviruses. Examples of retroviruses include mammalian C-type, B-type, D-type retroviruses, HTLV-BLV viruses, avian leukosis-sarcoma viruses (e.g., avian leukosis viruses, avian sarcoma viruses), lentiviruses and spumaviruses. Other examples include murine leukemia viruses, murine sarcoma viruses, mouse mammary tumor viruses, bovine leukemia viruses, feline leukemia viruses, feline sarcoma viruses, avian leukemia viruses, human T-cell leukemia viruses, baboon endogenous viruses, Gibbon ape leukemia viruses, Mason Pfizer monkey viruses, simian immunodeficiency viruses, simian sarcoma viruses, and Rous sarcoma viruses.

As herein defined, the terms “blocks” and “blocking”, when used herein, particularly in relation to viral entry, mean that a peptide expressed and displayed on the surface of phage or phagemid can bind to a ligand/binding molecule present on or attached to a cell membrane or indeed on the surface of an agent which interacts with a cell, for example the protein coat of a virus, wherein the binding member has a role in mediating viral entry into a cell, and the binding of the peptide can attenuate entry of the virus into a cell.

The term “peptide” is used herein to describe a series of at least two amino acids covalently linked by peptide bonds or modified peptide bonds such as isosteres. No limitation is placed on the maximum number of amino acids which may comprise a peptide.

As indicated above, the peptide expressed by the phagemid which binds to a ligand/binding molecule attached to a cell membrane may be an antibody. The term “antibody” should be construed as covering any molecule having a binding domain with the required specificity. The antibody of the invention may be a monoclonal antibody, or a fragment, derivative, functional equivalent or homologue thereof. The term includes any polypeptide comprising an immunoglobulin binding domain, whether natural or wholly or partially synthetic.

A “fragment” of a peptide of the invention, for example an antibody or a scFv is a shorter length of a contiguous sequence of the amino acids of said peptide. A derivative of a peptide of the invention, as identified by the method of the third aspect of the invention, means a peptide modified by varying the amino acid sequence of the peptide, e.g. by manipulation of the nucleic acid encoding the protein or by altering the protein itself. Such derivatives of the natural amino acid sequence may involve insertion, addition, deletion and/or substitution of one or more amino acids whilst still retaining function (ability to bind to binding member). Preferably such derivatives involve the insertion, addition, deletion and/or substitution of 25 or fewer amino acids, more preferably of 15 or fewer, even more preferably of 10 or fewer, more preferably still of 4 or fewer and most preferably of 1 or 2 amino acids only.

The expression “amino acid” as used herein is intended to include both natural and synthetic amino acids, and both D and L amino acids. A synthetic amino acid also encompasses chemically modified amino acids, including, but not limited to salts, and amino acid derivatives such as amides. Amino acids present within the polypeptides of the present invention can be modified by methylation, amidation, acetylation or substitution with other chemical groups which can change the circulating half life without adversely affecting their biological activity.

Nucleic acid for use in accordance with the present invention may comprise DNA or RNA and may be wholly or partially synthetic. In a preferred aspect, nucleic acid for use in the invention codes for antibodies or antibody fragments of the invention as defined above. The skilled person will be able to determine substitutions, deletions and/or additions to such nucleic acids which will still provide an antibody or antibody fragment of the present invention.

A peptide expressed on the surface of the phage can be a member of a library of peptides, for example a library of nucleotide sequences encoding VH and VL regions, which can encode scFvs.

Methods for the construction of phagemid antibody display libraries are well known in the art (for example, McCafferty et al. (1990) Nature 348 552-554. One particularly advantageous approach has been the use of scFv phage-libraries (see for example Huston et al., 1988, Proc. Natl. Acad. Sci USA).

### Administration

A peptide or a binding molecule/ligand identified by the methods of the present invention, may be administered alone, but will preferably be administered as a pharmaceutical composition, which will generally comprise a suitable pharmaceutically acceptable excipient, diluent or carrier selected depending on the intended route of administration. Examples of suitable pharmaceutical carriers include; water, glycerol, ethanol and the like.

A binding molecule, as identified by the first or second aspects of the invention, or a peptide of the invention, identified by the method of the third aspect of the invention, may be administered to a patient in need of treatment via any suitable route. Suitably, in particular embodiments, the composition is administered parenterally by injection or infusion. Examples of preferred routes for parenteral administration include, but are not limited to; intravenous, intracardial, intraarterial, intraperitoneal, intramuscular, intracavity, subcutaneous, transmucosal, inhalation or transdermal.

Routes of administration may further include topical and enteral, for example, mucosal (including pulmonary), oral, nasal, rectal.

The composition can be deliverable as an injectable composition. For intravenous, intradermal or subcutaneous application, the active ingredient can be in the form of a parenterally acceptable aqueous solution which is pyrogen-free and has suitable pH, isotonicity and stability. Those of relevant skill in the art are well able to prepare suitable solutions using, for example, isotonic vehicles such as sodium chloride injection, Ringer's injection or, Lactated Ringer's injection. Preservatives, stabilisers, buffers, antioxidants and/or other additives may be included, as required.

A composition comprising a peptide of the invention can also be administered via microspheres, liposomes, other microparticulate delivery systems or sustained release formulations placed in certain tissues including blood.

Examples of the techniques and protocols mentioned above and other techniques and protocols which may be used in accordance with the invention can be found in Remington's Pharmaceutical Sciences, 18th edition, Gennaro, A. R., Lippincott Williams & Wilkins; 20th edition ISBN 0-912734-04-3 and Pharmaceutical Dosage Forms and Drug Delivery Systems; Ansel, H. C. et al. 7th Edition ISBN 0-683305-72-7, the entire disclosure of which is herein incorporated by reference.

A composition comprising a binding molecule and/or peptide of the invention is preferably administered to an individual in a “therapeutically effective amount”, this being sufficient to show benefit to the individual to whom the composition is administered. The actual dose administered, and rate and time-course of administration, will depend on, and can be determined with due reference to, the nature and severity of the condition which is being treated, as well as factors such as the age, sex and weight of the patient to be treated and the route of administration. Prescription of treatment, e.g. decisions on dosage etc, is ultimately within the responsibility and at the discretion of general practitioners and other medical doctors.

Dosage regimens can include a single administration of the composition of the invention, or multiple administrative doses of the composition. The compositions can further be administered sequentially or separately with other therapeutics and medicaments which are used for the treatment of the condition for which the binding member and/or peptide of the present invention is being administered to treat.

Examples of dosage regimens which can be administered to a subject can be selected from the group comprising, but not limited to; 1 μg/kg/day through to 20 mg/kg/day, 1 μg/kg/day through to 10 mg/kg/day, 10 μg/kg/day through to 1 mg/kg/day.

Throughout the specification, unless the context demands otherwise, the terms ‘comprise’ or ‘include’, or variations such as ‘comprises’ or ‘comprising’, ‘includes’ or ‘including’ will be understood to imply the inclusion of a stated integer or group of integers, but not the exclusion of any other integer or group of integers.

As used herein, terms such as “a”, “an” and “the” include singular and plural referents unless the context clearly demands otherwise. Thus, for example, reference to “an active agent” or “a pharmacologically active agent” includes a single active agent as well as two or more different active agents in combination, while references to “a carrier” includes mixtures of two or more carriers as well as a single carrier, and the like.

Unless otherwise defined herein, scientific and technical terms used in connection with the present invention shall have the meanings that are commonly understood by those of ordinary skill in the art. Generally, nomenclatures used in connection with, and techniques of, cell and tissue culture, molecular biology, immunology, microbiology, genetics, protein and nucleic acid chemistry and hybridization described herein are those well known and commonly used in the art. The methods and techniques of the present invention are generally performed according to conventional methods well known in the art and as described in various general and more specific references that are cited and discussed throughout the present specification unless otherwise indicated. See, e.g., Sambrook et al., Molecular Cloning: A Laboratory Manual, 2nd ed., Cold Spring Harbor Laboratory Press, Cold Spring Harbor, N.Y. (1989) and Ausubel et al., Current Protocols in Molecular Biology, Greene Publishing Associates (1992), and Harlow and Lane, Antibodies: A Laboratory Manual, Cold Spring Harbor Laboratory Press, Cold Spring Harbor, N.Y. (1990), which are incorporated herein by reference.

### EXAMPLES

### Phage Display Library

The Tomlinson I library, phage KM13 (Kristensen and Winter, 1998), TG1 cells and an anti ubiquitin scFv (phagemid) were obtained from MRC HGMP resource centre (Cambridge).

Construction of Tomlinson I+J Libraries

These libraries are constructed in the pIT2 vector and comprise over 100 million different single chain variable fragments (scFv fragments) cloned into an ampicillin resistant phagemid vector and transformed into TG1 E. coli cells. scFv fragments comprise a single polypeptide with the V_{H} and V_{L} domains attached to one another by a flexible Glycine-Serine linker. The antibody libraries are expressed on KM13 particles which have a 21-residue peptide comprising a protease cleavage site, introduced into the flexible linker between the second and third domains of the minor coat protein pill of filamentous bacteriophage. The protease cleavage site allows the phage to become sensitive to cleavage using a range of proteases such as trypsin (Kristensen & Winter, 1998). Phagemids (Ab expressed phage) which bind to target molecule/s are eluted by trypsinization and amplified by infection into fresh TG1 E. Coli cells. All functional scFvs in both the I and J libraries bind to protein A and L.

2×TY Broth

16 g/L of tryptone, 10 g/L of yeast extract and 5 g/L of NaCl were dissolved, autoclaved and kept at +4° C.

TYE Agar

15 g/L of Bacto-agar, 8 g/L of NaCl, 10 g/L of tryptone and 5 g/L of yeast extract were dissolved, autoclaved and poured in bacterial grade petri dishes prior to being kept at +4° C.

YT Top Soft Agar

16 g/L of bacto-tryptone, 10 g/L of bacto-yeast extract, 7 g/L of bacto-agar and 5 g/L of NaCl were added to ddH₂O. The agar was heated to melting point and aliquoted in 50 ml volume prior to sterilization by autoclave.

Trypsin Stock Solution

Trypsin stock solution was made up by adding trypsin (Fluka) to 50 mM Tris-HCl (PH 7.4) and 1 mM CaCl₂ to obtain 10 mg/ml of stock concentration. The solution was stored at −20° C.

Trypsin-PBS Solution

10 μl of trypsin stock solution was added to 450 μl of PBS and stored at −20° C.

Bradford Reagent

100 mg of Coomassie Blue G-250 (Sigma) was dissolved in 50 ml of 95% (v/v) of ethanol mixed with 100 ml of 85% (w/v) of phosphoric acid. The mixture was made up to 1 L when the dye had completely dissolved and filtered through filter paper just before use.

1.5 M Tris-HCl, pH 8.8

121 g/L of tris base was added to ddH₂O. The pH was adjusted and the solution was stored at +4° C.

0.5 M Tris-HCl, pH 6.8

37.5 g/L of tris base was added to ddH₂O. The pH was adjusted and the solution was stored at +4° C.

Solubilizer Protein

250 mM of Tris-HCl pH 6.8 was added to 50 mM of SDS, 1.1 M of glycerol, 20 mM of DTT and 0.14 mM of bromophenol. The solution was kept at room temperature.

x1 Protein Buffer

This solution was prepared by adding 15 g/L of Tris base, 72 g/L of glycine and 5 g/L of SDS to ddH₂O and kept at room temperature.

Transfer Buffer

M of Tris was added to 192 mM of glycerine and 20% of methanol.

Cells and Viruses

Vero and SHSY5Y cells were grown in Dulbecco's Modified Eagle's Medium (DMEM)+GlutaMax-1 (GIBCO) supplied with 10% (v/v) of heat inactivated foetal bovine seyum (HI-FBS). For B95a cell, RPMI-1640 (Gibco) medium was supplemented with 5% (v/v) HI-FBS was used. All media were supplemented with 100 units/ml penicillin/streptomycin (Sigma). Maintenance medium (MM) was prepared similarly to GM, but supplemented with 1% (v/v) of HI-FBS and 100 units/ml penicillin/streptomycin. Confluent monolayers of Vero, SHSY5Y and B95a cells in 75 cm² flask were washed twice with 10 ml of PBS solution A. The PBS solution A was discarded and 2 ml of versene—for B95a) or trypsin/versene solution (other cell types) was added and the cells were incubated at 37° C. (except for B95a cell which was incubated at room temperature) for no longer than 5 minutes until they had detached from the flask. The appropriate growth media for all cell types was immediately added. The cell suspension was pipetted up and down several times to achieve a homogenous cell suspension prior to adding additional growth media. If necessary an aliquot of the cell suspension was counted using a haemocytometer to determine the cell concentration before cells were cultured by incubation at 37° C. in 5% v/v CO₂.

Cell Infection

SHSY5Y cells were grown on coverslips coated with 3% 3-aminopropyltriethoxysilane (APES-Sigma) and infected with WT Measles Virus (MV) at a MOI of 1 and fixed at selected dpi.

Immunofluorescence

Fixed coverslips were permeabilised and blocked. Indirect immunofluorescent was carried out using MAb anti-MV-N.

Production of KM13 helper phage

200 μl of TG1 at an OD₆₀₀ of 0.4 was infected with 10 μl of 100-fold serial dilutions of KM13 helper phage and incubated in a 37° C. water bath for 30 mins. The infected TG1 were then added to 3 ml of YT top soft agar and poured into warm TYE plates. The plates were allowed to set and incubated overnight at 37° C. The following day, a small plaque was picked and transferred into 5 ml of fresh TG1 at an OD₆₀₀ of 0.4 and incubated with shaking at 37° C. for 2 h. The culture was added to 500 ml of 2×TY and incubated with shaking at 37° C. for 1 h. Kanamycin (Sigma) was added to give a final concentration of 50 μg/ml. The bacterial culture was then incubated overnight with shaking at 30° C.

The overnight culture was centrifuged at 10,800 g for 15 minutes. 100 ml of PEG/NaCl (20% polyethylene glycol 600, 2.5 M NaCl) was added to 400 ml of supernatant and this was left for 1 h on ice. The culture was spun at 10,800 g for 30 minutes and the PEG/NaCl was removed. The pellet was then resuspended in 8 ml of PBS and 2 ml of PEG/NaCl was added. The mixture was placed on ice for 20 minutes. The culture was spun at 3,300 g for 30 minutes and the remaining PEG/NaCl was aspirated. The pellet was then resuspended in 5 ml of PBS and spun at 11,600 g for 10 minutes to remove any remaining bacterial debris. The helper phage was stored at +4° C. for short term storage or in PBS with 15% glycerol for longer time storage at −70° C.

Titration of KM13 Helper Phage

Helper phage were titred by adding 5 μl of trypsin stock solution to the 45 μl of phage and incubated for 30 minutes at 37° C. 1 μl of trypsin treated phage was serially diluted 10⁻¹ to 10⁻⁵ in PBS. 50 μl of each dilution (including 10⁰) of helper phage was added to six separate tubes containing 1 ml of TG1 at an OD₆₀₀ of 0.4. For each tube 3 ml of YT top soft agar was added and poured evenly onto TYE plates. Controls were performed using the same steps with non-trypsin treated phage.

Growing the Tomlinson I Library

500 μl of the library stock was added to 200 ml of 2×TY and grown with shaking at 37° C. until the (O)ₘ) reached 0.4. 2×10¹¹. Helper phage were added to 50 ml of the culture (the remaining culture was stored at −70° C.) and incubated in a water bath at 37° C. for 30 minutes. The culture was then centrifuged at 3000 g for 10 minutes and resuspended in 100 ml of 2×TY containing 100 μg/ml ampicillin, 50 μg/ml kanamycin and 0.1% glucose. Following overnight incubation at 30° C., the culture was centrifuged at 3,300 g for 30 minutes. 20 ml of PEG/NaCl was added to 80 ml of the culture and placed on ice for 1 h. The culture was then centrifuged at 3,300 g for 30 minutes and all the PEG/NaCl was removed. The pelleted cells were then resuspended in 4 ml of PBS and centrifuged at 11,600 g for 10 minutes to remove remaining bacterial debris. The phage were stored in PBS with 15% glycerol at −70° C.

The phage were titred by preparing a series of dilutions (10⁻¹ to 10⁻⁵) in 100 μl of PBS. 900 μl of TG1 (at OD₆₀₀ of 0.4) was added to each dilution (including) 10° and incubated at 37° C. in a water bath for 30 minutes. 10 μl from each dilution was plated on TYE plates containing 100 μg/ml ampicillin and 1% glucose. The plates were then incubated at 37° C. overnight and the number of colonies was counted.

Cell Membrane Preparation

A confluent monolayer of SHSY5Y cells was freeze thawed three times and centrifuged for 1000 g for 7 minutes. The supernatant was discarded and replaced by 4 ml of PBS. The membranes were centrifuged at 12,000 g for 5 minutes and this step was repeated 3 times, using fresh PBS each time. Finally, the membrane preparation was sonicated (MSE) for 2-3 minutes at +4° C. until the membrane clarified.

Protein Assay

The Bradford protein assay was carried out by diluting the cell membrane preparation to obtain between 5 to 100 μg protein and each assay tube containing 100 μl of sample. A standard was prepared using albumin (Sigma) at the same dilution. 5 ml of Bradford reagent was added to the samples and the standard before incubation for 5 minutes at room temperature. The absorbances were read at 595 nm.

Phage Selection

Immunotubes (Nunc) were coated overnight with 4 ng of the cell membrane preparation. The tubes were washed three times with PBS prior to incubation with 2% of MPBS (2 g of Marvel milk powder in 100 ml of PBS) at room temperature for 2 hrs for blocking. The tubes were then washed three times with PBS before adding 10¹² to 10¹³ phage in 4 ml of 2% MPBS and incubating for 60 minutes while rotating using an under-and-over turnover (Stuart Scientific) and then left standing for a further 60 minutes. The supernatant was discarded and the tube was washed with PBS containing 0.1% of Tween 20 (10 times for the first selection and 20 times for further selections). Phage were eluted by adding 500 μl of trypsin-PBS and rotating for 10 minutes using an under-and-over turnover. The eluted phage were stored at +4° C.

Preparation of Bio-Assay Plates

250 μl of eluted phage was added to 1.75 ml of TG1 at OD 600 of 0.4 and incubated for 30 minutes in a 37° C. water bath. 10 μl of 10⁻² and 10⁻⁴ dilutions of phage were spotted onto TYE plates containing 100 μg/ml ampicillin and 1% glucose. The plates were incubated overnight at 37° C. On the following day, the colonies were counted to obtain the titre of the phage produced from each round of selection.

For each round of selection, the remaining TG1 cultures were centrifuged at 11,600 g for 5 minutes to remove bacterial debris before resuspending the pelleted bacteria in 1 ml of 2×TY and then plating on a large square Bio-Assay dish (Nunc) containing TYE agar with 100 μg/ml of ampicillin and 1% glucose. The plates were grown overnight at 37° C.

Production of Phagemids

After overnight growth, 7 ml of 2×TY containing 15% glycerol was added to the Bio-Assay dish. The cells were mixed thoroughly using a glass spreader. 50 μl of the scraped bacteria was added to 50 ml of 2×TY containing 100 μg/ml of ampicillin and 1% glucose, while the rest was stored in 1 ml aliquots at −70° C.

The bacteria were grown by shaking at 37° C. until an OD₆₀₀ of 0.4 was obtained. 5×10¹⁰ of the helper phage were added to 10 ml of the culture. The culture was then incubated in a water bath at 37° C. for 30 minutes and centrifuged at 3000 g for 10 minutes. The pellet was resuspended in 50 ml of 2×TY containing 100 μg/ml of ampicillin, 0.1% of glucose and 50 μg/ml of kanamycin. The culture was incubated overnight with shaking at 30° C. On the following day, the culture was spun at 3,300 g for 15 minutes. 10 ml of PEG/NaCl was added to 40 ml of supernatant, mixed and left on ice for 1 h. The culture was then spun at 3,300 g for 30 minutes and respun briefly (for a few seconds) to remove all the remaining PEG/NaCl. The pellet was resuspended in 2 ml of PBS and centrifuged at 11,600 g for 10 minutes to remove the remaining bacterial debris. This phage stock was stored at +4° C. and used for the next round of selections as summarized in FIG. 1.

Virus Infection and Blocking Assay

After the final round of selections, bacterial colonies were picked from the bio-assay plate and pools of 20-30 of these made. Production of phagemids was carried out as previously described. The phagemids were then used in virus blocking assays.

100 μl of each phagemid pool was added to 80-90% confluent SHSY5Y cells grown in 96 well plates and incubated at +4° C. for 45 minutes. WT MV at a dilution of 10⁻² and 10⁻³ (titre of the virus used was 10^{5.1} TCID₅₀/ml) were added to each of four wells. The plates were incubated at 37° C. in 5% CO₂ for 7 days and the CPE monitored. Phagemid pools which produced inhibition of virus infection were grown up, subdivided into smaller numbers of colonies (by returning to the original bioassay plate) and the phagemids were produced as described herein. These selection steps were repeated until a single colony which blocked the WT MV infection was obtained. In all blocking experiments the ubiquitin specific phagemid was used as a control.

Results

Phagemid Blocking WT MV Infection

Using the display phage library approach a phagemid was obtained following subdividing of phagemid pools 4 times. This phagemid was referred to as 4.7.3.1 and resulted in the blocking of WT MV infection in SHSY5Y cells at 24 and 48 hpi for both dilutions of virus used (FIG. 2a & b). No CPE or MV protein expression was detected by immunofluorescence using a monoclonal antibody to the nucleocapsid (N) protein of MV when the 4.7.3.1 phagemid was used in the assay. In contrast in SHSY5Y cells treated with the anti-ubiquitin control phagemid, (FIG. 2c) were positive for antigen staining at 48 hours post infection and showed a similar level of infection to the untreated controls (FIG. 2d). Furthermore, other phagemids in the sub library selected to bind to the SHSY5Y cells did not block infection and therefore acted as an internal control i.e. phagemids which bind to cells but do not block infection indicating that they recognise molecules on the cell membrane but these do not act as receptors for WT MV. A further method was used to confirm that phagemid 4.7.3.1. blocked virus replication. RNA was extracted from cells treated with 4.7.3.1. or treated with the control phagemids or with no phagemid (as described above) and inoculated with WT MV. Non-infected cells were also used as a control. The RNA was reverse transcribed using an oligo dT primer to ensure that virus mRNA (i.e. not virus genomic RNA) was specifically transcribed. The cDNA was then amplified by PCR using specific MV primers. PCR products were detected for cells inoculated with WT MV but either not treated with phagemid or treated with a control phagemid. In contrast, no PCR products were observed when cells were treated with phagemid 4.7.3.1. and then inoculated with virus. These results indicate that phagemid 4.7.3.1 directly blocks a receptor used for cell entry in SHSY5Y cells rather a molecule having an effect at a later stage during the virus replication cycle.

Using the identified phagemid 4.7.3.1, the binding molecule to which the peptide displayed on this phage binds can be identified and characterised. A number of approaches are possible for selecting the binding molecule.

1.) Phagemid 4.7.3.1 can be used to stain bands on a Western blot of solubilised proteins in the cell membrane preparation. If the same band can be stained with Comassie blue and visualised there should be enough protein to excise the band and carry out mass spectrometry to identify the molecule. As will be appreciated other identification and/or characterisation methods may be utilised as known in the art.

2. Phagemid 4.7.3.1. can be used to immunoprecipitate the binding molecule from the membrane preparation. The immunoprecipitate complex can then be run on a gel to separate the binding molecule. As above Comassie blue staining is required to determine if there is sufficient protein for analysis.

3. An affinity column can be prepared with phagemid 4.7.3.1. Solublised membrane protein is run through the column. The binding protein can then be eluted in suitable buffer conditions and analysed by, for example, mass spectrometry to identify the molecule. This third method may be used if sufficient quantities of the binding molecule are not obtained by methods 1 and 2.

As will be appreciated by those of skill in the art, any suitable methods may be used to identify and characterise binding molecules or binding members with binding specificity to said binding molecules. Suitably, where mass spectrometry is used, once suitable spectrometry data has been obtained, this data may be provided to an appropriate search engine which can compare the data to protein sequence databases to match peptide sequences with the mass spectra data and based on this suggest candidate binding molecules/binding members for consideration. Such methods are presently utilised in, for example, systems biology

Although the invention has been particularly shown and described with reference to particular examples, it will be understood by those skilled in the art that various changes in the form and details may be made therein without departing from the scope of the present invention.

### REFERENCES

Boder, E. T., Midelfort, K. S. & Wittrup, K. D. (2000). Direct evolution of antibody fragments with monovalent femtomolar antigen-binding affinity. Proceedings of the National Academy of Sciences USA. 97: 10701-10705.
Boder, E. T. & Wittrup, K. D. (1997). Yeast surface display for screening combinatorial polypeptide libraries. National Biotechnology. 15: 553-557
Jermutus, L., Honegger, A., Schwesinger, F., Hanes, J. & Pluckthun, A. (2001). Tailoring in vitro evolution for protein affinity or stability. Proceedings of the National Academy of Sciences USA. 98: 75-80.
Hanes, J., Jermutus, L., Weber-Bornhauser, S., Bosshard, H. R. & Pluckthun, A. (1998). Ribosome display efficiently selects and evolves high-affinity antibodies in vitro from immune libraries. Proceedings of the National Academy of Sciences USA. 95: 14130-14135.
Hanes, J. & Pluckthun, A. (1997). In vitro selection and evolution of functional proteins by using ribosome display. Proceedings of the National Academy of Sciences USA. 94: 4937-4942.
He, M. & Taussig, M. J. (1997). Antibody-ribosome-mRNA (ARM) complexes as efficient selection particles for in vitro display and evolution of antibody combining sites. Nucleic Acids Research. 25: 5132-5134.
Hoogenboom, H. R. (2005). Selecting and screening recombinant antibody libraries. Nature Biotechnology. 23: 1105-1116.
Protein engineering of antibody binding sites: recovery of specific activity in an anti-digoxin single-chain Fv analogue produced in Escherichia coli. Proc Natl Acad Sci USA. 1988 August; 85(16):5879-83
Huston J S, Levinson D, Mudgett-Hunter M, Tai M S, Novotny J, Margolies M N, Ridge R J, Bruccoleri R E, Haber E, Crea R, et al. (1988). Protein engineering of antibody binding sites: recovery of specific activity in an anti-digoxin single-chain Fv analogue produced in Escherichia coli. Proc Natl Acad Sci USA. 16:5879-83
Lipovsek, D. & Pluckthun, A. (2004). In vitro protein evolution by ribosome display and mRNA display. Journal of Immunology Methods. 290: 51-67.
Mattheakis, L. C., Bhatt, R. R. & Dower, W. J. (1994). An in vitro polysome display system for identifying ligands from very large peptide libraries. Proceedings of the National Academy of Sciences USA. 91: 9022-9026.
McCafferty, J., Griffiths, A. D., Winter, G. & Chiswell, D. J. (1990). Phage antibody: filamentous phage display antibody variable domains. Nature. 348: 552-554.
Naniche, D., Wild, T. F., Rabourdin-Combe, C. & Gerlier, D. (1992). A monoclonal antibody recognizes a human cell surface glucoprotein involved in measles virus binding. Journal of General Virology. 73: 2617-2624.
O'Connell, D., Becerril, B., Roy-Burman, A., Daws, M. & Marks, J. D. (2002). Phage versus phagemid libraries for generation of human monoclonal antibodies. Journal of Molecular Biology. 321: 49-56.
Poul, M. A. & Marks, J. D. (1999). Targeted gene delivery to mammalian cells by filamentous bacteriophage. Journal of Molecular Biology. 288: 203-211.

What is claimed is:

1. A method for identifying a binding molecule provided by a test surface, the method comprising the steps of:
providing a test surface;
providing at least one phage expressing at least one displayed peptide on the phage surface;
exposing the test surface to the at least one phage under conditions which permit binding of a displayed peptide to a binding molecule on the test surface when the displayed peptide has binding specificity to a binding molecule on the test surface;
contacting the test surface with an agent which interacts with the test surface via the unknown binding molecule provided by the test surface wherein said agent can be detected by reporter means,
determining the level of the agent bound to the test surface using the reporter means;
wherein, when the level of the agent bound to the test surface in the presence of a phage is decreased relative to a control, it is indicative that the phage is bound to a binding molecule which mediates binding to the agent,
using the peptide of the bound phage to select the binding molecule provided by the test surface to which the phage binds and
identifying said binding molecule.

2. The method as claimed in claim 1 wherein the reporter means include, a fluorescent reporter molecule or a radioactive label provided on the agent or an antibody capable of binding to the agent.

3. The method as claimed in claim 1 wherein the reporter means is a detectable biological event, wherein the method comprises the steps of:
providing a test surface;
providing at least one phage expressing at least one displayed peptide on the phage surface;
exposing the test surface to the at least one phage under conditions which permit binding of a displayed peptide to a binding molecule provided by the test surface when the displayed peptide has binding specificity to the binding molecule provided by the test surface;
contacting the test surface with an agent which interacts with the test surface via an unknown binding molecule and causes a biological event,
determining the level of the biological event;
wherein, when the level of the biological event in the presence of a phage is decreased relative to a control, it is indicative that the phage is bound to the binding molecule which mediates the biological event,
using the peptide provided on the phage to select the binding molecule provided by the test surface to which the phage binds and
identifying said binding molecule.

4. The method of claim 3 wherein the biological event includes cell migration, differentiation, apoptosis, cell proliferation, virus replication or the like.

5. A method as claimed in claim 1 wherein the agent is selected from a protein, a proteinaceous moiety, a lipid, a carbohydrate, a virus or portion thereof, a bacterium or portion thereof, or fungi or a portion thereof.

6. The method as claimed in claim 1 for identifying an unknown binding molecule present on an infective agent or a cell capable of being infected, the method comprising the steps of:
providing a test surface including an unknown binding molecule which mediates infection,
exposing the test surface to at least one phage wherein the phage has at least one displayed peptide on the phage surface,
providing conditions which permit binding of a displayed peptide to a binding molecule of the test surface when the displayed peptide has binding specificity to a binding molecule of the test surface;
providing conditions which allow infection,
determining the level of infection,
wherein, when the level of infection in the presence of the phage is decreased relative to a control, it is indicative that the phage is bound to a binding molecule which mediates infection,
using the displayed peptide to select the binding molecule in or on the test surface to which the phage binds and
identifying said binding molecule.

7. The method of claim 1 further comprising the step of identifying a binding member with binding specificity to a binding molecule as identified by claim 1.

8. A method for identifying a peptide with binding specificity to an unknown binding molecule provided by a test surface, wherein said method comprises the steps of:
providing a test surface,
providing at least one phage expressing at least one displayed peptide on the phage surface;
exposing the test surface to the at least one phage under conditions which permit binding of a displayed peptide to a binding molecule provided by the test surface when the displayed peptide has binding specificity to a binding molecule provided by the test surface;
contacting the test surface with an agent which interacts with the test surface via an unknown binding molecule wherein said agent can be detected by reporter means,
determining the level of the agent bound using the reporter means;
wherein, when the level of the reporter means in the presence of a phage is decreased relative to a control, it is indicative that the phage is bound to the unknown binding molecule, and
identifying the peptide displayed by the phage with binding specificity to the binding molecule of the test surface.

9. The method according to claim 1 wherein the displayed peptide is an antibody or a fragment thereof.

10. The method according claim 9 wherein the antibody is provided by a library constructed from at least one of:
(i) Ig variable region genes derived from the B cells of an immunized donor;
(ii) B cells of a non-immunized donor;
(iii) a combination of germline V genes and synthetic oligonucleotide sequences encoding random CDRs
(iv) a fully synthetic library; and
(v) the Tomlinson I and J phage display library.

11. A method according to claim 1 wherein the method further comprises characterising at least one of the identified unknown binding molecule and a peptide displayed by a phage with binding specificity to said unknown binding molecule.

12. An assay to identify modulating agents which specifically bind to a binding molecule comprising the steps:
contacting a peptide with binding specificity to the unknown binding molecule as identified using the method of claim 7 in the absence of a putative modulating agent under conditions which would allow binding between the displayed peptide and the unknown binding molecule;
contacting a peptide with binding specificity to the unknown binding molecule in the presence of a putative modulating agent under conditions which would allow binding between the displayed peptide and the unknown binding molecule in the absence of the putative modulating agent, and
determining the level of binding of the peptide in the presence and absence of the putative modulating agent,
wherein when the putative modulating agent decreases the level of binding of the displayed peptide to the binding molecule, the putative modulating agent is considered to have binding specificity to the unknown binding molecule.

13. A method of inhibiting infection of a cell by a virus, wherein the method comprises administering to a cell a binding molecule or peptide as identified in claim 1.

14. A method of treating a disease in a subject, said method comprising administering to said subject a binding molecule or peptide as identified by the method of claim 1.

15. (canceled)

16. (canceled)

17. A kit for use in the method methods as claimed in claim 1 comprising a sub-library of phage displaying peptides with binding specificity to the binding molecules provided on a selected cell type or tissue preparation.

18. The kit as claimed in claim 17 wherein the selected cell type can include at least one of neuronal cells, NT2 cells, neuroblastoma cells, SHSY5Y cells, Vero cells, epithelial cells, mouse cells, rat cells, or human cells.
